(19) United States
(12) Patent Application Publication
(10) Pub. No.: US 2026/0224096 A1
(43) Pub. Date: Aug. 6, 2026

(54) INSERTION TUBE FOR A MEDICAL DEVICE WITH SLOT PATTERNS

(71) Applicant: Boston Scientific Scimed, Inc., Maple Grove, MN (US)

(72) Inventors: Sean POWELL, Holden, MA (US); Gene Thomas STORBECK, Millville, MA (US)

(73) Assignee: Boston Scientific Scimed, Inc., Maple Grove, MN (US)

(21) Appl. No.: 19/465,409

(22) Filed: Jan. 30, 2026

Related U.S. Application Data
(60) Provisional application No. 63/753,106, filed on Feb. 3, 2025.

(51) Int. Cl. A61B 1/005; A61B 1/00

(52) U.S. Cl. CPC A61B 1/0055; A61B 1/0011

(57) ABSTRACT

An insertion tube for a medical device shaft including a plurality of semi-continuous slots arranged in a helical pattern along a length of the insertion tube. The insertion tube including a plurality of intervals separating the slots. The intervals comprising a plurality of helical intervals extending circumferentially, and the helical intervals may be circumferentially offset such that the plurality of slots are staggered. The intervals may further comprise a plurality of longitudinal intervals extending along the length of the insertion tube, and the longitudinal intervals may define a longitudinal space between the plurality of slots.

### CROSS-REFERENCE TO RELATED APPLICATIONS

This application claims the benefit of priority to U.S. Provisional Application No. 63/753,106, filed on February 3, 2025, which is incorporated by reference herein in its entirety.

### TECHNICAL FIELD

Various embodiments of this disclosure relate generally to insertion tubes for medical devices, for example, medical devices having insertion tubes with various slot patterns.

### BACKGROUND

During a medical procedure, a user may operate a medical device that includes a handle and a shaft extending therefrom. The shaft may terminate in a distal tip portion that incorporates features such as optical elements (e.g., cameras, lighting), air/water outlets, and working channel openings for medical tools. Some devices also incorporate a steering mechanism, which helps to allow the user to deflect or adjust the orientation of the distal tip of the shaft of the medical device. For example, a user may move or otherwise manipulate the shaft by actuating pull wires or control members.

Shafts may undergo forces during delivery and be configured to help transmit mechanical force/energy from the pull wires or control members. Medical procedures may benefit from shafts configured to maintain or improve the ability to effectively transmit forces (e.g., torques), while balancing torsional stiffness, axial stiffness, bending flexibility, etc. during delivery.

### SUMMARY

Aspects of the disclosure relate to, among other things, devices and methods related to insertion tubes with slots or features (e.g., patterned slots or features) that help the shaft of a medical device deflect, move, or otherwise perform a procedure. Each of the aspects disclosed herein may include one or more of the features described in connection with any of the other disclosed aspects.

In one example, an insertion tube for a medical device shaft may include a plurality of semi-continuous slots arranged in a helical pattern along a length of the insertion tube. The insertion tube may also include a plurality of intervals separating the slots. The intervals may comprise a plurality of helical intervals extending circumferentially. The helical intervals may be circumferentially offset such that the plurality of slots is staggered. The intervals may further comprise a plurality of longitudinal intervals extending along the length of the insertion tube, and the longitudinal intervals may define a longitudinal space between the plurality of slots.

Any examples described herein may have any of these features alone or in combination. The slots may be arranged as a plurality of pairs of slots along the length of the insertion tube and are substantially parallel. Each slot of the pairs of slots may be spaced apart longitudinally by longitudinal intervals, and each slot of the pairs of slots may be offset by helical intervals. Each pair of a plurality of pairs of slots may be aligned in the same direction. Helical intervals may separate each pair of the plurality of pairs of slots circumferentially, such that the plurality of pairs of slots is staggered around a circumference of the insertion tube. The slots may be arranged in alternating directions along the length of the insertion tube. A first group of slots may be aligned in a first direction, and a second group of slots may be aligned in a second direction that is opposite the first direction. A third group of slots may be aligned in the first direction or the second direction. Helical intervals may separate the first group of slots from the second group of slots. A plurality of compressive load paths may be formed along a torsional axis of the insertion tube, and the compressive load paths are directed along the helical pattern of the slots. A plurality of tensile load paths may intersect the compressive load paths along the torsional axis. The tensile load paths may be directed along the helical pattern of the slots in a direction opposite the compressive load paths. A plurality of nodes may be formed at intersections of the compressive and tensile load paths. A plurality of non-continuous load paths may be formed along the length of the insertion tube, and the non-continuous load paths may be interrupted by the plurality of slots. The insertion tube may further comprise a reinforcement layer applied to the insertion tube, and the reinforcement layer includes at least one of a braid or coil. The insertion tube may further comprise an outer layer coating. The outer layer coating may include at least one of an extruded coating, a film wrap, or a laminated layer.

In another example, a medical device shaft may comprise an insertion tube for a medical device shaft, and the insertion tube may include a plurality of semi-continuous slots arranged in a helical pattern along a length of the insertion tube.

The insertion tube may also include a plurality of intervals separating the plurality of slots. The intervals may comprise a plurality of helical intervals extending circumferentially. The helical intervals may be circumferentially offset such that the plurality of slots are staggered. The intervals may further comprise a plurality of longitudinal intervals extending along the length of the insertion tube. The longitudinal intervals may define a longitudinal space between the plurality of slots. The insertion tube may also include a plurality of continuous load paths along the torsional axis, and the plurality of continuous load paths may form intersecting helices along the insertion tube. A plurality of nodes may be formed at intersections between continuous load paths.

Any of the examples disclosed herein may include any of the following features in any combination. A plurality of non-continuous load paths may be formed along the length of the insertion tube, and the non-continuous load paths may be interrupted by the plurality of slots. A first group of slots may be aligned in a first direction, and a second group of slots may be aligned in a second direction opposite the first direction.

In another example, a method of forming an insertion tube is provided. The method may comprise positioning a tube in a manufacturing apparatus including a plurality of rotating heads. The method may include simultaneously cutting a plurality of helical slots into the insertion tube using the rotating heads. The rotating heads may include indented surfaces to form uncut portions between the plurality of helical slots.

In some aspects of the disclosure, the method may include one or more of the following features. The tube may comprise a first material and a second material. After the helical slots are formed, the method may further include heating the slotted tube to a temperature above a melting point of the first material but below a melting point of the second material.

It may be understood that both the foregoing general description and the following detailed description are exemplary and explanatory only and are not restrictive of the disclosure, as claimed.

### BRIEF DESCRIPTION OF THE DRAWINGS

The accompanying drawings are incorporated herein and constitute a part of this specification, illustrate exemplary aspects of the disclosure, and together with the description, explain the principles of the disclosure.

FIG. 1 illustrates an exemplary medical device, including a handle and a shaft;

FIG. 2 illustrates an exemplary insertion tube that may be used with the exemplary medical device of FIG. 1, according to aspects of this disclosure;

FIG. 3 illustrates an insertion tube with different slot patterns, according to aspects of this disclosure;

FIG. 4 illustrates another insertion tube with different slot patterns, according to aspects of this disclosure;

FIG. 5 illustrates another insertion tube with different slot patterns, according to aspects of this disclosure; and

FIG. 6A illustrates another insertion tube with different slot patterns, and FIG. 6B illustrates various force paths transmitted through the insertion tube of FIG. 6A, according to aspects of this disclosure;

FIG. 7 illustrates another exemplary insertion tube, according to aspects of this disclosure;

FIGS. 8A-8B illustrate an exemplary system used to form an exemplary insertion tube, according to aspects of this disclosure; and

FIG. 9 is a block diagram depicting an exemplary method of forming an exemplary insertion tube, according to aspects of this disclosure.

### DETAILED DESCRIPTION

Aspects of the disclosure include medical devices with shafts including one or more insertion tubes and methods of manufacturing insertion tubes with slots or features (e.g., patterned slots or features) that help transmit movement or otherwise impart forces, for example, to a distal portion of the shaft of the medical device. The ability to deflect the distal portion of a shaft helps a user position the distal portion of the shaft relative to a treatment site more efficiently, which may help to reduce procedure time, increase patient safety, help a user grasp/manipulate tissue, etc. Accordingly, the devices and methods disclosed herein may help result in better patient outcomes.

The medical device may be a medical device with a shaft, which may be a catheter, a scope (endoscope, bronchoscope, colonoscope, duodenoscope, etc.), a tube, a sheath, or other like device, capable of being inserted into a body cavity or lumen, for example the GI tract, via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any natural opening, any other body tract, or any bodily incision.

Furthermore, wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. Proximal and distal directions are labeled with arrows marked “P” and “D”, respectively, throughout various figures.

As used herein, the terms “comprises,” “comprising,” or any other variation thereof, are intended to cover a non-exclusive inclusion, such that a process, method, article, or apparatus that comprises a list of elements does not necessarily include only those elements but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/- 10% of a stated value. As used herein, the phrase “based on” is understood to be equivalent to the phrase “based at least on,” unless indicated otherwise. The term “or” is used disjunctively, such that “at least one of A or B” includes, (A), (B), (A and A), (A and B), (B and B), etc.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Any structures of the medical devices described herein can be made of biocompatible materials, including biocompatible polymers, rubbers, plastics, and the like.

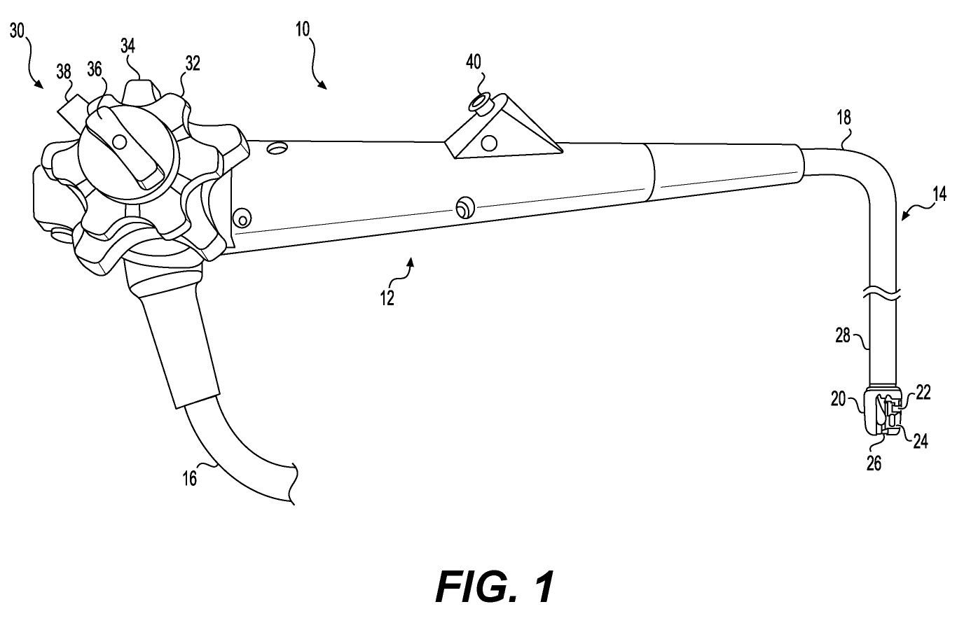

Reference will now be made in detail to aspects of the disclosure, examples of which are illustrated in the accompanying drawings. Embodiments of this disclosure relate to aspects of an insertion tube with slots or features (e.g., patterned slots or features) configured to help transmit or otherwise impart movement or forces to a distal portion of a medical device. FIG. 1 illustrates aspects of a medical device, including a handle and a shaft.

FIG. 1 illustrates an exemplary duodenoscope 10 having a handle 12 and a shaft or an insertion portion 14. As discussed in detail below and as shown in other Figures, insertion portion 14 includes at least one insertion tube, which includes slots or features (e.g., patterned slots or features). Medical procedures may benefit from insertion tubes with a steerable section that balances torsional stiffness for steerability, axial stiffness for pushability, and bending stiffness for flexibility.

In the art, torsional stability may be achieved by incorporating coiled ribbons made of metal or plastic as a base layer. However, this multi-material and layered approach to forming insertion tubes may require integrating different materials through different manufacturing techniques. In the art, the flexibility of single-piece metal or plastic insertion tubes may also be improved by introducing horizontal slots (e.g., resembling ladder rungs) along the length of an insertion tube. Horizontal slots may enhance directional flexibility, allowing for improved bending movement. However, horizontal slots may not always enhance the tube's ability to transmit torque.

Aspects of this disclosure describe various embodiments of insertion tubes configured to maintain or improve the ability to effectively transmit torque while balancing torsional stiffness, axial stiffness, and bending flexibility. As described herein, the insertion tubes may include slots or features arranged in a generally helical pattern that may help distribute or transfer mechanical loads along the length and/or circumference of the insertion tubes. In some aspects, the helical pattern may help to enhance torque transfer, which can help facilitate precise control of the distal portion of a medical device during procedures.

Duodenoscope 10 may also include an umbilicus 16 for purposes of connecting duodenoscope 10 to sources of, for example, air, water, suction, power, etc., as well as to image processing and/or viewing equipment. Although a duodenoscope may be referenced herein, it will be appreciated that the disclosure also encompasses endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a shaft 18 and a distal tip 20. Shaft 18 may extend from a distal portion of handle 12 and may be a sufficient length to reach target sites within the body of a subject. Distal tip 20 may include an imaging device 22 (e.g., a camera) and a lighting source 24 (e.g., an LED or an optical fiber). Distal tip 20 may be side-facing, such that imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device (not shown) may face distally (approximately parallel to a longitudinal axis of shaft 18).

Distal tip 20 may also include an elevator 26 for changing an orientation of a tool inserted in a working channel of duodenoscope 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26. A distal portion of shaft 18 that is connected to distal tip 20 may have a steerable section 28. Handle 12 may have one or more actuators/control mechanisms 30. Control mechanisms 30 may provide control over steerable section 28 or may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28.

Handle 12 may further include one or more locking mechanisms 36 (e.g., knobs or levers) for preventing steering and/or braking of steerable section 28 in at least one of an up, down, left, or right direction. Handle 12 may include an elevator control lever 38, which may raise and/or lower elevator 26, via connection between lever 38 and an actuating wire that extends from lever 38, through shaft 18, to elevator 26.

A port 40 may allow delivery or passage of a tool, fluid or suction source, etc. through port 40, into a working channel of the duodenoscope 10, through shaft 18, to distal tip 20. In use, an operator may insert at least a portion of shaft 18 into a body lumen of a subject. Distal tip 20 may be navigated to a target site in the body lumen. The operator may insert an accessory device (not shown) into port 40 and pass the accessory device through shaft 18 via a working channel to distal tip 20. The accessory device may exit the working channel at distal tip 20. The user may use elevator control lever 38 to raise elevator 26 and angle the accessory device toward a desired location (e.g., a papilla of the pancreatico-biliary tract).

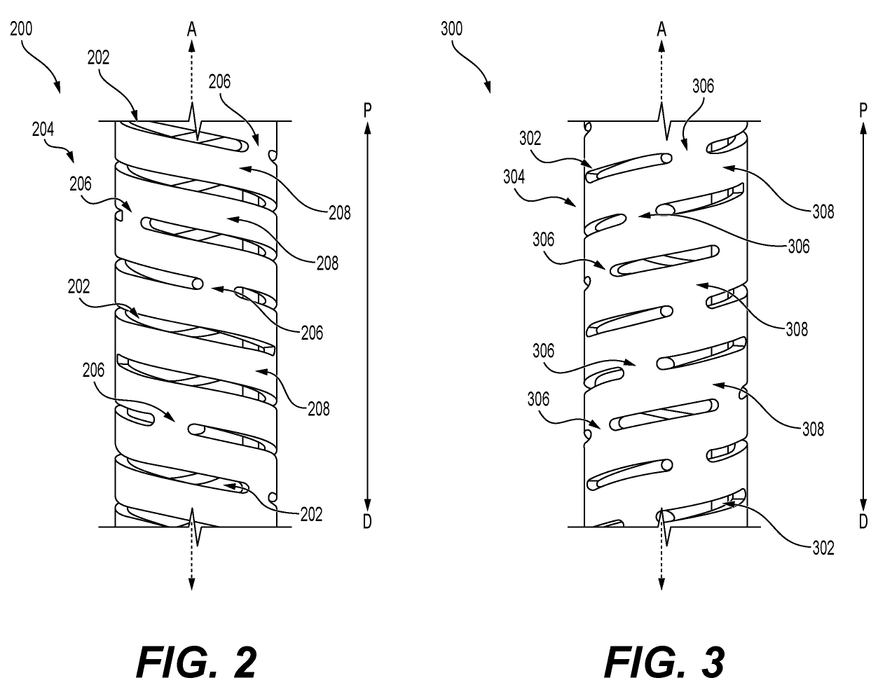

FIG. 2 illustrates an insertion tube 200 that may be used in medical procedures, as described above. Insertion tube 200 or any insertion tube described herein may be used with the medical device of FIG. 1. For example, insertion tube 200 may be incorporated (e.g., as a layer) in shaft 18 of duodenoscope 10 (FIG. 1). Insertion tube 200 may include a plurality of slots 202, which may be arranged in a generally helical pattern along the length of insertion tube 200. For example, each of the slots 202 may extend from a proximal direction to a distal direction along the length of insertion tube 200. Slots 202 may be spaced apart or spaced apart by a plurality of intervals 204.

Intervals 204 may be uncut or otherwise non-manipulated portions of insertion tube 200 that separate each of the plurality of slots 202. Intervals 204 may include helical intervals 206 and longitudinal intervals 208 (e.g., along axis A), one or more of which may contribute to the mechanical properties of insertion tube 200. Helical intervals 206 may include uncut portions of insertion tube 200 that extend circumferentially between adjacent slots 202, while longitudinal intervals 208 may include uncut portions of insertion tube 200 that extend axially or longitudinally between adjacent slots 202. The overall slot pattern, defined by slots 202 and intervals 204, may form a semi-continuous configuration of insertion tube 200. In some aspects, slots 202 may be longer compared to alternative configurations shown in FIGS. 3-6. As shown in other embodiments, the length and arrangement of slots 202 and intervals 204 may be adjusted to achieve one or more of desired flexibility, torsional stability, or force transmission characteristics for insertion tube 200.

Longitudinal intervals 208 may correspond to uncut portions of insertion tube 200 that represent the distance between successive slots 202 along the length of the insertion tube 200. Each longitudinal interval 208 may define a spacing or pitch of slots 202 in the longitudinal direction. Helical intervals 206 may correspond to uncut portions of the insertion tube 200 separating the plurality of slots 202 circumferentially or helically. In some aspects, helical intervals 206 may be circumferentially offset, which may help form a staggered arrangement of slots 202 rather than alignment of slots 202 along a single direction or plane of insertion tube 200. This staggered configuration may help distribute forces along the length of insertion tube 200, which may help to transmit torsional loads while maintaining flexibility.

FIG. 3 illustrates an insertion tube 300 that may be used with a medical device (e.g., medical device 10), in accordance with examples of this disclosure. As shown in FIG. 3, insertion tube 300 is similar to insertion tube 200. For example, insertion tube 300 may include a plurality of slots 302, which may be arranged in a generally helical pattern along the length of insertion tube 300. Slots 302 may be spaced apart or spaced apart by a plurality of intervals 304, which may include a plurality of helical intervals 306 and a plurality of longitudinal intervals 308.

However, in some aspects, slots 302 may be shorter in length compared to the slots 202 in the insertion tube 200 shown in FIG. 2. As a result, the helical pattern of slots 302 may be more interrupted, for example, compared to the pattern of slots 202. For example, each slot of the plurality of slots 302 may cover less surface area along the circumference of insertion tube 300 compared to the plurality of slots 202 shown in FIG. 2. Moreover, the orientation of the slot pattern in the insertion tube 300 may be reversed or otherwise in a different direction compared to, for example, the insertion tube 200 shown in FIG. 2. For example, slots 302 may extend from a distal direction to a proximal direction along the length of insertion tube 200.

Helical intervals 306 may be uncut portions that separate the plurality of slots 302 circumferentially around the insertion tube 300. In some aspects, helical intervals 306 may be more frequent compared to helical interval 206 of the insertion tube 200 due to the shorter length and/or amount of slots 302, which may lead to a more interrupted helical pattern. The increased number of helical intervals 306 may help the torsional resistance of insertion tube 300. For example, by providing additional structural integrity through increased surface area of uncut portions and preventing deformation of insertion tube 300. Longitudinal intervals 308 may represent the distance between successive slots 302 along the length of the insertion tube 300. Longitudinal intervals 308 may define the pitch of the helical pattern and, in combination with the helical intervals 306, may result in a staggered slot arrangement.

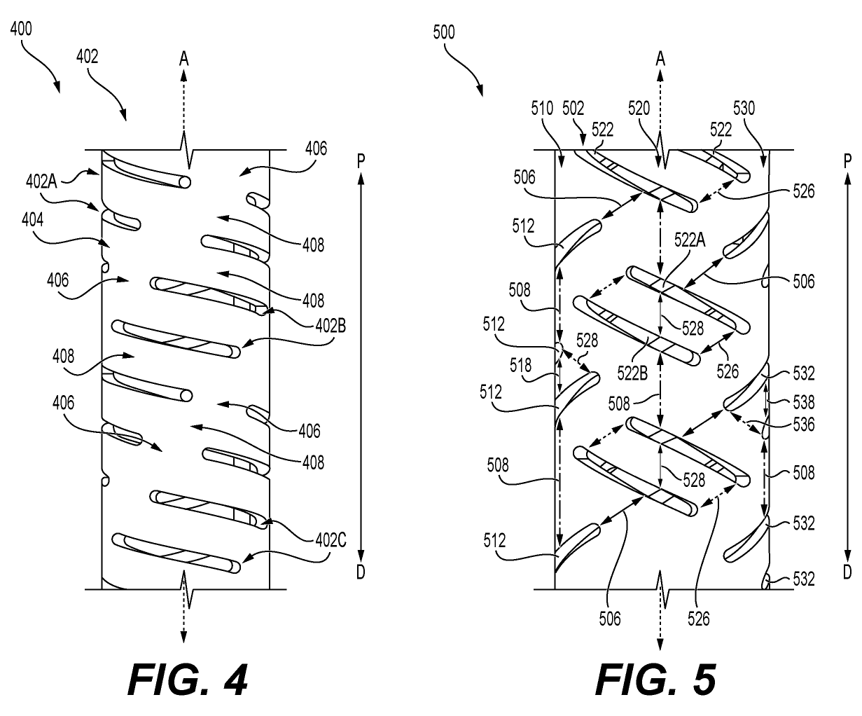

FIG. 4 illustrates an insertion tube 400 that may be used with a medical device (e.g., medical device 10), in accordance with examples of this disclosure. Insertion tube 400 may include a plurality of slots 402 arranged in a generally helical pattern along the length of insertion tube 400 (e.g., proximal to distal). Each of the plurality of slots 402 may be adjacent to one another and spaced apart by a plurality of intervals 404, which may include helical intervals 406 and longitudinal intervals 408. As discussed below, slots 402 may be spaced apart by intervals 404 such that slots 402 are circumferentially offset to form a helical arrangement of slots along insertion tube 400.

Slots 402 may be organized into groups formed along insertion tube 400. For example, slots 402 may include a first slot group 402A, a second slot group 402B, a third slot group 402C, etc. In some aspects, the arrangement shown in FIG. 4 includes groups each with two slots, but this disclosure may encompass other configurations. For example, some embodiments may include groupings of slots that include three slots, four slots, or a different number. In some aspects, each group of the plurality of slots 402 may be close to parallel along the length of insertion tube 400. It is noted that not all of the plurality of groups of slots are labeled in FIG. 4.

Each group of slots 402 may be aligned in the same direction. For example, plurality of slots 402 may extend and align in the same direction (e.g., clockwise), while each adjacent slot may be spaced apart both longitudinally and helically/circumferentially. As shown in FIG. 4, first slot group 402A and second slot group 402B, may extend in a same direction, which may form a uniform pattern along the length of the insertion tube 400. Additional slot groups, such as third slot group 402C and subsequent groups, may extend along insertion tube 400 in a consistent directional alignment.

Helical intervals 406 may separate the plurality of slots 402, such that the slots are staggered around the circumference of insertion tube 400. For example, second slot group 402B may be circumferentially offset or staggered relative to the first slot group 402A. This staggered configuration may help enhance torsional stability of insertion tube 400, for example, by distributing slots across multiple planes. The staggered arrangement formed by helical intervals 406 may help reduce excessive deformation in specific directions. Longitudinal intervals 408, on the other hand, represent the spacing between successive rows of slot pairs along the length of the insertion tube. For example, second slot group 402B is longitudinally offset from first slot group 402A.

Each slot within a slot group (e.g., first slot group 402A) may also be spaced apart by a longitudinal interval and a helical interval, which together may define the spacing between the slots within the pair. These intervals are described in more detail in connection with the embodiment shown in FIG. 5. For example, FIG. 5 illustrates a helical interval 526 and a longitudinal interval 528 that may separate slot 522A from slot 522B. In some aspects and referring back to FIG. 4, consistent with the embodiments of FIGS. 2 and 3, each pair of slots 402 may also be spaced apart by intervals 404, which may include both helical intervals 406 and longitudinal intervals 408.

FIG. 5 illustrates an insertion tube 500 that may be used with a medical device (e.g., medical device 10), in accordance with examples of this disclosure. Similar to insertion tube 400, insertion tube 500 includes a plurality of slots 502 arranged in a generally helical pattern along its length. As shown in FIG. 5, slots 502 may be organized in substantially parallel pairs along the length of the insertion tube and may be spaced apart both longitudinally and circumferentially. However, unlike the configuration in FIG. 4, circumferentially spaced pairs of slots 502 may be aligned in opposing (e.g., perpendicular) directions.

In some aspects, slots 502 may be organized into groups based on the direction in which slots 502 extend. For example, a first group of slots 510 may include a plurality of pairs of 512, each aligned and extended in a first direction from a proximal to distal direction of insertion tube 500 (e.g., elongating clockwise). A second group of slots 520 may include a plurality of pairs of slots 522, each aligned and extending in a second, opposing direction (e.g., counterclockwise). A third group of slots 530 may include a plurality of pairs of slots 532, which may extend in a similar or alternating direction. Although not shown, slots 502 may include a fourth group of slots, for example, on the rear of insertion tube 500 in FIG. 5, and the fourth group of slots may extend in the same direction as second group of slots 520. Moreover, each respective slot within each group may be spaced apart by a longitudinal interval and a helical interval, which may define the spacing and orientation of the slots within the group.

For example, in the first group of slots 510, each slot of the plurality of pairs of slots 512 may be spaced apart by a longitudinal interval 518 or distance that separates the slots in a longitudinal direction of insertion tube 500 (e.g., along axis A). Additionally, each slot of the plurality of pairs of slots 512 may be spaced apart by a helical interval 516 that separates each slot helically. Similarly, in the second group of slots 520, each slot of the pair of slots 522 may be spaced apart by a longitudinal interval 528 and a helical interval 526. The third group of slots 530 may include plurality of pairs of slots 532 with spacing defined by a longitudinal interval 538 and a helical interval 536.

Similar to FIG. 4, the embodiment of FIG. 5 includes a plurality of helical intervals 506 that helically separate an entire group of slots, such that the slots are staggered around the circumference of insertion tube 500. For example, plurality of pairs of slots 512 of the first group of slots 510 may be helically offset or staggered relative to plurality of slots 522 of the second group of slots 520. Due to the alternating directions of the plurality of pairs of slots (e.g., 512, 522, and 532), helical intervals 506 may exhibit a more staggered and irregular pattern compared to the embodiment shown in FIG. 4. This staggered configuration may help prevent the alignment of slots within a single plane, and may help to minimize deformation of insertion tube 500 in one or more directions. Longitudinal intervals 508, on the other hand, define the spacing between successive rows of plurality of pairs of slots along the length of the insertion tube within the same group. For example, a longitudinal interval 508 separates each pair of slots 512 in the first group of slots 510 from other pairs of slots 512 in the first group of slots 510. As discussed, longitudinal intervals may contribute to the pitch of the slot pattern and may play a role in balancing flexibility, rigidity, and torsional resistance.

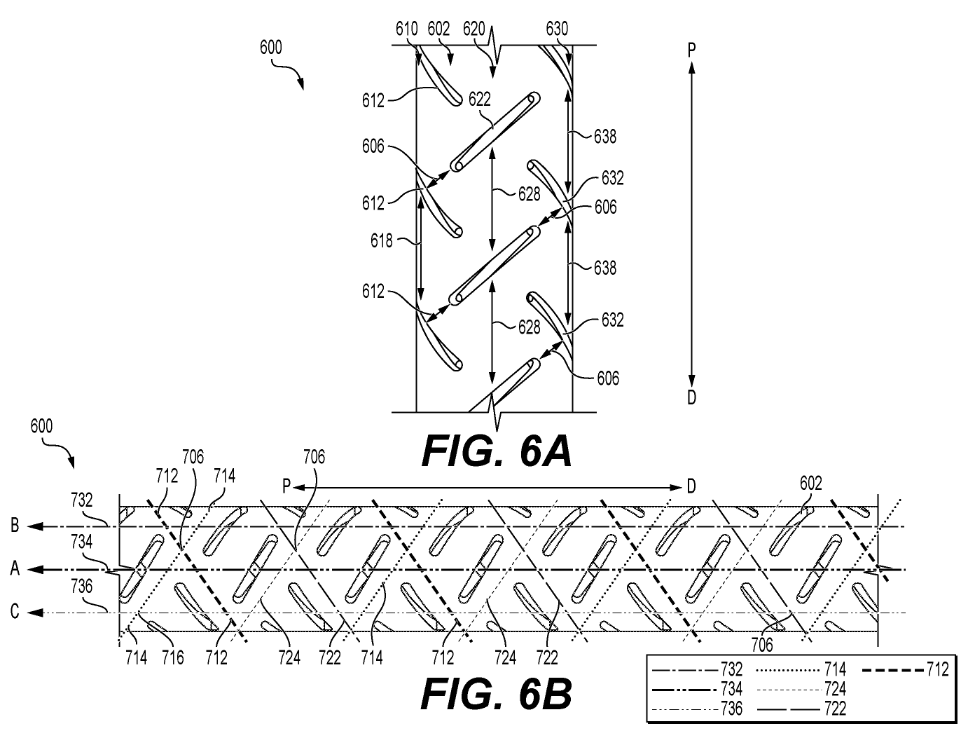

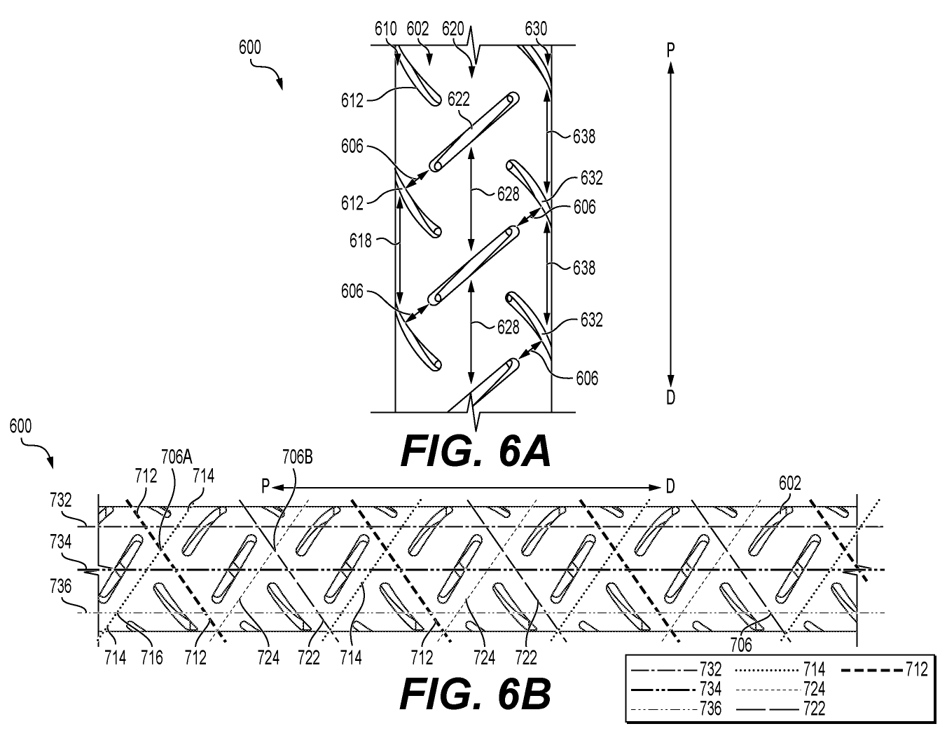

FIG. 6A illustrates an insertion tube 600 that may be used with a medical device (e.g., medical device 10), in accordance with examples of this disclosure. Similar to insertion tube 500, insertion tube 600 includes a plurality of slots 602 arranged in a generally helical pattern along its length. These slots 602 are spaced apart by a plurality of intervals 604, which may include helical intervals 606 and longitudinal intervals 608. Unlike the paired slot arrangement shown for insertion tube 400 or insertion tube 500, insertion tube 600 includes a pattern of single slots arranged sequentially along the length of insertion tube 600.

As shown in FIG. 6A, each group of slots 602 may be oriented in an alternating direction. For example, a respective slot 602 may extend in a clockwise direction followed by another slot 602 elongating in a counterclockwise direction. Slots 602 may be grouped based on the direction in which they extend. For example, a first group of slots 610 may include a plurality of slots 612, which may be aligned and extend in a first direction. In some aspects, each individual slot 612 in first group of slots 610 may extend in a clockwise direction from a proximal end to a distal end of insertion tube 600. A second group of slots 620 may include a plurality of slots 622, each aligned and elongating in a second, opposing direction (e.g., counterclockwise). A third group of slots 630 may include a plurality of slots 632, which may extend in a similar or alternating direction relative to the previous groups. Although not shown, slots 602 may include a fourth group of slots, for example, on the rear of insertion tube 600 in FIG. 6A, and the fourth group of slots may extend in the same direction as second group of slots 620.

Each respective slot within each group may be spaced apart by a longitudinal interval, which may define the spacing and orientation of the slots within the group. For example, in the first group of slots 610, each slot 612 may be spaced apart from other slots 612 in the first group of slots 610 by a longitudinal interval 618 or distance. Similarly, in the second group of slots 620, each slot 622 may be spaced apart from other slots 622 in the second group of slots 620 by a longitudinal interval 628 or distance. The third group of slots 630 may include a plurality of slots 632, and each slot 632 may be spaced apart from other slots 632 in the third group of slots 630 by a longitudinal interval 638.

Similar to FIG. 5, the embodiment of FIG. 6A may include a plurality of helical intervals 606 that separate each group of plurality of pairs of slots or adjacent plurality of pairs of slots helically, such that slots may be staggered around the circumference of insertion tube 600. For example, slots 612 of the first group of slots 610 may be helically offset or staggered relative to slots 622 of the second group of slots 620. Due to the alternating directions of slots (e.g., 612, 622, and 632), helical intervals 606 may exhibit a staggered and irregular pattern similar to the design shown in FIG. 5. As will be described in FIG. 6B, exemplary helical patterns may help to provide benefits for insertion tubes during medical procedures, and the effect of the helical pattern will be described in more detail below.

FIG. 6B illustrates insertion tube 600, according to aspects of this disclosure. As described above, insertion tube 600 may include a plurality of slots 602, which may be arranged in an angled helical configuration along the circumference and length of insertion tube 600. FIG. 6B illustrates various load paths that may be formed by the structure of insertion tube 600 and other insertion tubes discussed herein. While insertion tube 600 is shown, any insertion tube described in this disclosure may have similar properties. The load paths may help to illustrate how mechanical loads may be distributed and transferred along the length and circumference of insertion tubes during a medical procedure. In some aspects, the staggered, offset alignment of slots 602 may form load paths that are distributed along multiple helical areas of insertion tube 600 rather than concentrated along a single plane.

FIG. 6B illustrates various load paths, including one or more compressive load paths 712 and 722 and one or more tensile load paths 714 and 724, representing load paths formed around the axis of insertion tube 600 when subjected to a torsional load. Load paths 712 and 722 may represent areas of insertion tube 600 that experience compressive or tensile forces when insertion tube 600 is subjected to torsional loads in alternate directions. Load paths 714 and 724 may represent areas of insertion tube 600 that experience tensile or compressive forces opposite the magnitude of load paths 712 and 722 when insertion tube 600 is subjected to the same torsional loads. In some aspects, longitudinal intervals 618, 628, and 638 and helical intervals 606 between slots 602 may help direct the compressive forces along the helical pattern of slots 602, which may help maintain uniform torsional stiffness throughout insertion tube 600.

For example, load paths 712 and 722 may help direct forces along insertion tube 600 such that insertion tube 600 may resist a degree of torsional or twisting forces, which may help insertion tub 600 to retain its steerability during medical procedures. Load paths 714 and 724, representing continuous tensile load paths, may complement load paths 712 and 722. Like the compressive load paths, tensile load paths 714 and 724 may be directed along the helical pattern of slots 602, which may help insertion tube 600 resist stretching or elongation under tension and contribute to the axial stiffness of insertion tube 600. This may aid in the ease of advancement of the insertion tube 600 through a body lumen during medical procedures.

Compressive load paths 712 and 722 and tensile load paths 714 and 724 may intersect along the length of insertion tube 600, forming a geometry that may resemble crisscrossing helices or "X-shaped" intersections. The compressive load paths 712 and 722 may spiral helically in one direction (e.g., clockwise). In contrast, the tensile load paths 714 and 724 may spiral in the opposite direction (e.g., counterclockwise) and intersect at intervals, which may help form a plurality of nodes 706A, 706B, etc. A plurality of nodes 706A and 706B may be formed at the intersections of the compressive and tensile load paths. This intersecting geometry may create a braided or woven appearance, with nodes 706A, 706B potentially representing connection points for continuous load paths, forming a pattern that may function similarly to a braid, except that continuous load paths may be bonded rather than interwoven. Nodes 706A, 706B may serve as transfer points between different load-bearing elements, which may help enhance the overall structural integrity of the insertion tube 600.

In addition to the continuous load paths, insertion tube 600 may include a plurality of non-continuous load paths 732, 734, and 736. Non-continuous load paths 732, 734, and 736 may represent areas where the load transfer is partially disrupted or intermittent due to slots 602. For example, non-continuous load paths 732, 734, and 736 may be formed along the length of insertion tube from a proximal to distal direction where slots 602 interrupt the transfer of forces along a particular direction. Non-continuous load paths 732, 734, and 736 may create regions of increased flexibility or articulation points along the length of insertion tube 600. Each non-continuous load path may extend along a different plane of insertion tube 700. For example non-continuous load path 734 may extend along a central longitudinal axis of insertion tube 700. Therefore, non-continuous load paths 732, 734, and 736 may help to form regions of increased flexibility or articulation points along multiple planes or axes (e.g., along longitudinal axes A, B, and C) of insertion tube 700 rather than articulation points concentrated along a single plane.

In some aspects, non-continuous load paths 732, 734, and 736 may be formed in desired positions to help the maneuverability of the insertion tube based on the requirements of a medical procedure. For example, the pattern of slots 602 may be modified, which may in turn modify the load paths. In some cases, adjusting the pitch or spacing of slots 602 and intervals 604 helices may help balance of flexibility and rigidity for specific medical applications, which may help insertion tube 600 be effective for use in various medical procedures.

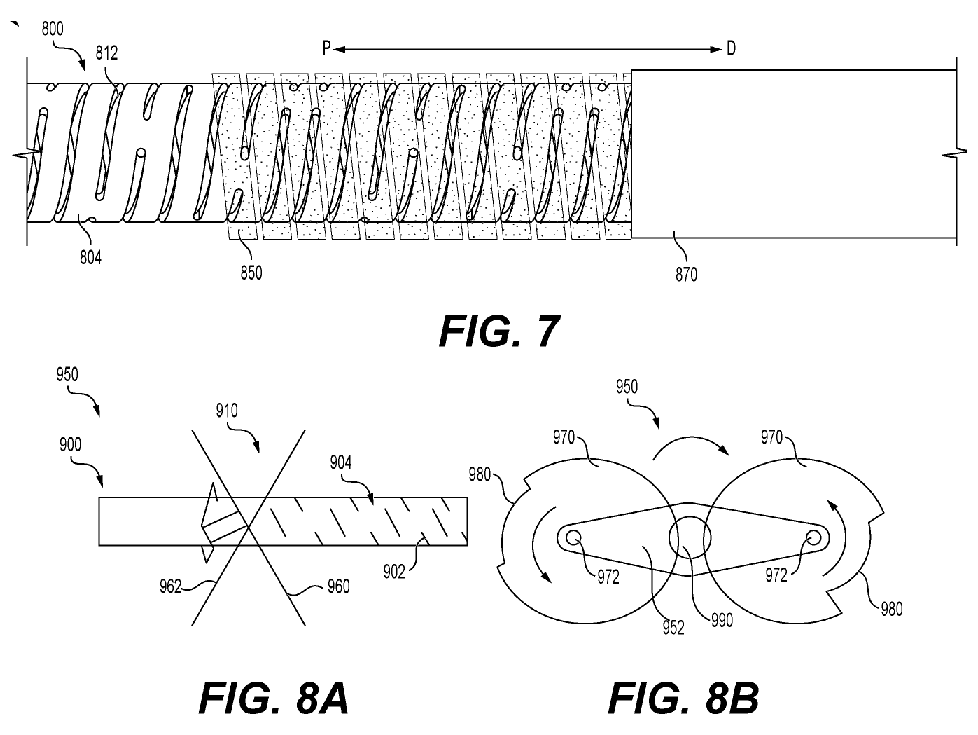

FIG. 7 illustrates portions of an exemplary shaft 810 that may be used with a medical device (e.g., medical device 10), including insertion tube 800, according to aspects of this disclosure. Insertion tube 800 may be similar to insertion tube 200, shown in FIG. 2, except as described herein. Insertion tube 800 may include a plurality of slots 812. For example, insertion tube 800 may include a plurality of slots 802, which may be arranged in a generally helical pattern along the length of insertion tube 800. Similar to insertion tubes discussed above, slots 802 may be spaced apart or spaced apart by a plurality of intervals 804, which may include a plurality of intervals 804 (e.g., longitudinal and helical intervals). In some aspects, insertion tube 800 may include additional features that may optionally be incorporated into any of the insertion tubes discussed in this disclosure.

As discussed above, the pattern of helical slots of insertion tubes discussed herein may distribute mechanical loads along the length and circumference of insertion tubes. In some aspects, shaft 810 may include one or more reinforcement layers 850, which may help to increase the stiffness or strength of shaft 810. For example, as shown in FIG. 7, insertion tube 800 may be at least partially surrounded by a reinforcement layer 850, which may be incorporated to form shaft 810. Alternatively, although not shown, reinforcement layer 850 may be radially within insertion tube 800 to form shaft 810. In any of these examples, reinforcement layer 850 may include braiding or coiling. A braided reinforcement may help to increase the torsional rigidity of the tube, potentially providing additional resistance to twisting forces. Alternatively, a coiled reinforcement may be included in regions of shaft 810 where additional pushability or axial stiffness may be helpful.

Shaft 810 may also include an outer layer coating 870. In some aspects, the outer layer coating 870 may be an extruded coating applied to the outer surface of shaft 810, for example, exterior to reinforcement layer 850. Alternatively, outer layer coating 870 may be a film wrap or lamination, which may be used to apply a thin, protective layer in the formation of shaft 810. Outer layer coating 870 may help enhance abrasion resistance or improve the ability of shaft 810 to withstand external forces during use, for example, increased resistance to environmental conditions like moisture or chemicals. The combination of reinforcement layer 850 and outer layer coating 870 may allow further customization of the properties of insertion tube 800, and thus shaft 810, to suit specific medical applications.

FIGS. 8A-8B illustrate a system for manufacturing an insertion tube 900 with a plurality of helical slots 902. Insertion tube 900 may be similar to any of the insertion tubes discussed herein. FIG. 8A depicts a manufacturing apparatus 910 used to form insertion tube 900 including helical slots 902 spaced apart by a plurality of intervals 904, which may include helical and longitudinal intervals. Cut path 960 indicates a predetermined and adjustable pattern to create helical slot 902 in insertion tube 900. In some aspects, a second cut path 962 may be used to form slots in another direction (e.g., as shown in FIGS. 5, 6A, and 6B). FIG. 8B shows a cutting mechanism 950 comprising a support member 952, and one or more rotating heads, for example, two rotating heads 970 that rotate in opposite directions, as the curved arrows indicate. In some aspects, rotating heads 970 may be configured with various cutting mechanisms, such as cutting tools, lasers, or end mills.

Support member 952 may include one or more mounting points 972 for attaching rotating heads 970. As shown in FIG. 8B, support member 952 may include two mounting points 972 to couple two rotating heads 970 to support member 952. Support member 952 may also include a connecting point 990 that may be positioned between rotating heads 970. Connecting point 990 may help to coordinate the movement of rotating heads 970, which may help synchronize rotation during the use of cutting mechanism 950. In some aspects, each rotating head 970 may have an indented surface 980 along its outer edge. Indented surface 980 may be a non-cutting section, which allows areas of insertion tube 900 to remain intact, forming intervals. The structure of rotating heads 970 may thus regulate which areas of insertion tube 900 receive cuts, allowing for intermittent helical slots 902 to be formed. The rotating heads 970 may transition simultaneously along insertion tube 900 to form or cut helical slots 902 into the insertion tube. As rotating heads 970 rotate, they may follow cut paths 960 to form the slots while maintaining intervals 904 between cuts. In some aspects, adjusting parameters like the rotation speed, cutting depth, or spacing of rotating heads 970 may allow customization of the slot pattern to achieve desired mechanical characteristics for specific medical applications. Modifying these parameters may provide flexibility in tailoring the insertion tube properties to suit various procedural requirements.

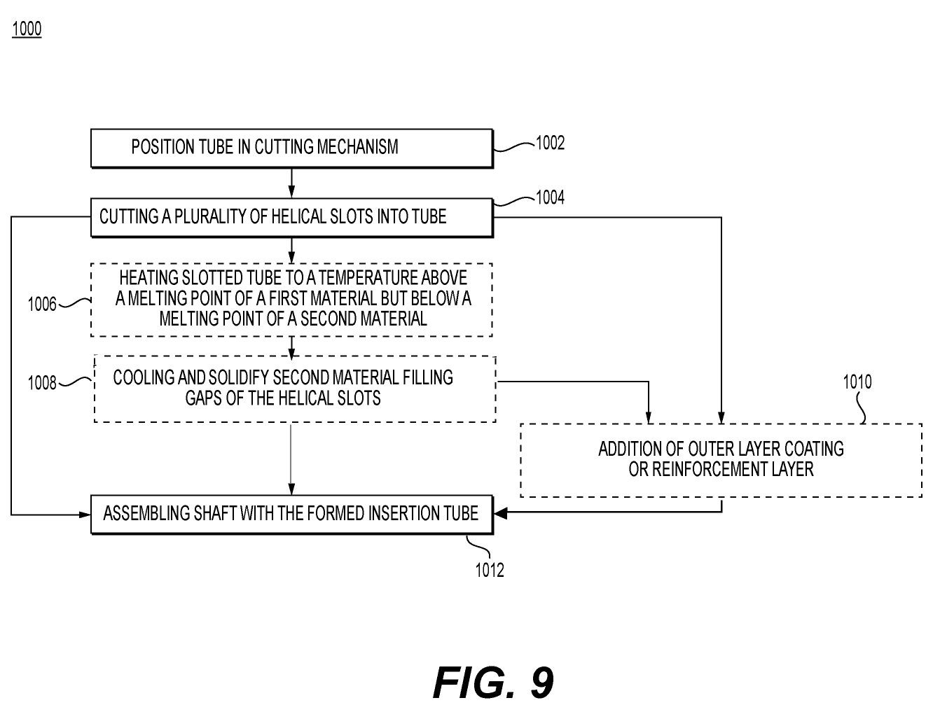

FIG. 9 illustrates a flow chart of an exemplary method 1000 for manufacturing an insertion tube of a medical device. The method may begin with an initial insertion tube or tube blank that serves as the base structure. This tube blank may be formed from a single material, such as metal or polymer, or a combination of materials (e.g., two materials as described below), depending on the desired mechanical properties. Method 1000 includes a first step 1002, which includes positioning the tube blank in a cutting mechanism (e.g., the cutting mechanism 950 shown in FIGS. 8A and 8B).

Step 1004 includes cutting a plurality of helical slots into the tube blank. During step 1004, the tube blank may be rotated and moved longitudinally along cutting mechanism 950 relative to rotating heads 970. In some aspects, rotating heads 970 transition longitudinally relative to the tube blank. This relative longitudinal movement, by the tube blank, rotating heads 970, or both, may help form slots at desired angles and orientations along the cut paths 960 and/or 962. For example, longitudinal movement may occur such that rotating heads 970 follow cut path 960 and/or 962 to form slots at desired angles and orientations. The rotating heads 970 include indented surfaces 980, which allow uncut portions of the tube to remain intact, forming longitudinal and helical intervals between slots. In some aspects, a user may adjust the pitch of helical slots to tailor the balance of flexibility and rigidity. The user may also modify the slot length, orientation, or spacing to control the insertion tube's bending, torsional, or axial stiffness. The pattern of helical slots may distribute mechanical loads along the length and circumference of insertion tubes. In some cases, this distribution of loads may reduce or eliminate using a core or mandrel typically used to reinforce the tube cross-section and prevent deformation during manufacturing.

In some aspects, the initial blank insertion tube may comprise more than one material, for example, a first material or an inner elastomeric layer and a second material or an outer elastomeric layer, forming a two-material tube. An optional step 1006 may include heating the slotted tube to a temperature above the melting point of the first material but below a melting point of the second material. During the heating, molten elastomeric material may flow into the cut slots, at least partially filling the gaps of the slots. Similarly, an optional step 1008 may follow to cool and solidify the material filling the gaps of the slots. This process may result in a tube structure where the new material bridges the gaps in the slots. In some cases, an optional step 1010 may be performed after step 1004 or step 1008. Step 1010 may involve applying one or more additional outer layer(s) or reinforcement layer(s), as discussed in relation to FIG. 7. The method may conclude with step 1012. Step 1012 include assembling a shaft for a medical device with the formed insertion tube, which may include any necessary post-processing operations, such as trimming or attaching components of a distal tip (e.g., distal tip 20 in FIG. 1).

Various aspects discussed herein may help improve insertion tubes of medical devices and potentially increase efficacy in treating a treatment site. The helical slot patterns of insertion tubes may help to distribute forces and enhance torsional stability. Furthermore, the methods described may provide a way to form various embodiments of insertion tubes. Accordingly, insertion tubes discussed herein may allow for improved maneuverability and control during medical procedures.

While principles of this disclosure are described herein with reference to illustrative aspects for various applications, it should be understood that the disclosure

is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

What is claimed is:

1. An insertion tube for a medical device shaft, comprising:
a plurality of semi-continuous slots arranged in a helical pattern along a length of the insertion tube; and
a plurality of intervals separating the slots, the intervals comprising:
a plurality of helical intervals extending helically, wherein the helical intervals are offset such that the plurality of slots is staggered;
a plurality of longitudinal intervals extending along the length of the insertion tube, wherein the longitudinal intervals define a longitudinal space between the plurality of slots.

2. The insertion tube of claim 1, wherein the slots are arranged as a plurality of pairs of slots along the length of the insertion tube and are substantially parallel.

3. The insertion tube of claim 2, wherein each slot of the pairs of slots are spaced apart longitudinally by longitudinal intervals, wherein each slot of the pairs of slots are offset by helical intervals.

4. The insertion tube of claim 3, wherein each pair of a plurality of pairs of slots are aligned in a same direction.

5. The insertion tube of claim 3, wherein helical intervals separate each pair of the plurality of pairs of slots circumferentially, such that the plurality of pairs of slots are staggered around a circumference of insertion tube.

6. The insertion tube of claim 1, wherein the slots are arranged in alternating directions along the length of the insertion tube.

7. The insertion tube of claim 6, wherein:
a first group of slots is aligned in a first direction, and
a second group of slots is aligned in a second direction that is opposite the first direction.

8. The insertion tube of claim 7, wherein a third group of slots is aligned in the first direction or the second direction.

9. The insertion tube of claim 7, wherein helical intervals separate the first group of slots from the second group of slots.

10. The insertion tube of claim 1, wherein a plurality of compressive load paths is formed along a torsional axis of the insertion tube, and wherein the compressive load paths are directed along the helical pattern of the slots.

11. The insertion tube of claim 10, wherein a plurality of tensile load paths intersects with the plurality of compressive load paths along the torsional axis, wherein the tensile load paths are directed along the helical pattern of the slots in a direction opposite to the compressive load paths.

12. The insertion tube of claim 11, wherein a plurality of nodes is formed at intersections between the compressive and tensile load paths.

13. The insertion tube of claim 12, wherein a plurality of non-continuous load paths is formed along the length of the insertion tube, and wherein the non-continuous load paths are interrupted by the slots.

14. The insertion tube of claim 1, further comprising a reinforcement layer applied to the insertion tube, wherein the reinforcement layer includes at least one of a braid or coil.

15. The insertion tube of claim 1, further comprising an outer layer coating, wherein the outer layer coating includes at least one of an extruded coating, a film wrap, or a laminated layer.

16. A medical device shaft, comprising:
an insertion tube, wherein the insertion tube includes;
a plurality of slots arranged in a generally helical pattern along a length of the insertion tube;
 a plurality of intervals separating the plurality of slots, the intervals comprising:
a plurality of helical intervals extending circumferentially, wherein the helical intervals are circumferentially offset such that the plurality of slots is staggered;
a plurality of longitudinal intervals extending along the length of the insertion tube, wherein the longitudinal intervals define a longitudinal space between the plurality of slots; and
a plurality of continuous load paths along a torsional axis, wherein the plurality of continuous load paths form intersecting helices along the insertion tube, wherein a plurality of nodes is formed at intersections between continuous load paths.

17. The medical device shaft of claim 16, wherein a plurality of non-continuous load paths is formed along the length of the insertion tube, wherein the non-continuous load paths are interrupted by the plurality of slots.

18. The medical device shaft of claim 17, wherein,
a first group of slots is aligned in a first direction, and
a second group of slots is aligned in a second direction that is opposite the first direction.

19. A method of forming an insertion tube, comprising:
positioning a tube in a cutting mechanism including a plurality of rotating heads; and
cutting a plurality of helical slots into the tube using the rotating heads, wherein the rotating heads include indented surfaces to form uncut portions between the plurality of helical slots.

20. The method of forming insertion tube of claim 19, wherein the tube comprises a first material and a second material, wherein after the helical slots are formed, the method further includes heating the slotted tube to a temperature above a melting point of the first material but below a melting point of the second material.
